(19) United States
(12) Patent Application Publication
(10) Pub. No.: US 2025/0295720 A1
(43) Pub. Date: Sep. 25, 2025

(54) DESIGNED MACROALGAE FEED PRODUCTS

(71) Applicants: Alliance for Sustainable Energy, LLC, Golden, CO (US); Ocean Era, Inc., Kailua-Kona, HI (US)

(72) Inventors: Lieve M.L. LAURENS, Denver, CO (US); Robert Scott NELSON, Arvada, CO (US); Neil Anthony SIMS, Kailua-Kona, HI (US); Simona AUGYTE, Kailua-Kona, HI (US)

(21) Appl. No.: 18/843,564

(22) PCT Filed: Mar. 1, 2023
(86) PCT No.: PCT/US2023/063473

§ 371 Date: Sep. 3, 2024

Related U.S. Application Data
(60) Provisional application No. 63/315,332, filed on Mar. 1, 2022.

(51) Int. Cl. A61K 36/04; A23K 10/12; A23K 10/30; A23K 50/80; A61K 9/00; A61K 36/03; A61K 36/05; A61P 3/02

(52) U.S. Cl. CPC A61K 36/04; A23K 10/12; A23K 10/30; A23K 50/80; A61K 9/0056; A61K 36/03; A61K 36/05; A61P 3/02

(57) ABSTRACT

Disclosed herein are methods and compositions for the microbiological pretreatment of macroalgal biomass, to create an alternative, low cost, environmentally friendly, scalable and sustainable source of key nutrients for aquaculture and live-stock industries. In an embodiment, disclosed herein is an aquatic feedstuff and ingredient quality that has no dependence on wild fishery or terrestrial resources, and in addition provides a fish-health and productivity benefit to the fish. This feed quality benefit is thought to extend to additional feed formulations, e.g. livestock (poultry, swine, etc.).

### CROSS-REFERENCE TO RELATED APPLICATION

This application is a national phase entry under 35 U.S.C. § 371 and claims priority to PCT application number PCT/US2023/063473 filed 1 Mar. 2023 which claims priority under 35 U.S.C. § 119 to U.S. provisional patent application No. 63/315,332 filed on Mar. 1, 2022, the contents of which are hereby incorporated in their entirety.

### CONTRACTUAL ORIGIN

The United States Government has rights in this invention under Contract No. DE-AC36-08GO28308 between the United States Department of Energy and the Alliance for Sustainable Energy, LLC, the Manager and Operator of the National Renewable Energy Laboratory.

### BACKGROUND

Decarbonizing agriculture and aquaculture feed formulations requires a focus around creating ingredients that consist of lower carbon intensity compared to traditional ingredients.

Very limited information is available on the inclusion of macroalgae as feed ingredients. Typically, inclusion rates are limited because of the recalcitrant nature of the biomass and the high salt content.

Seaweeds have traditionally been used as a source of fodder for livestock diets in coastal regions, such as in Ireland. Others have reviewed recent interest in use of macroalgae as ingredients for various animal agriculture feeds. Some species of seaweeds (e.g. Asparagopsis taxiformis) may offer potential to reduce methane emissions when included in ruminant diet formulations. Seaweeds may also offer chelated minerals, polyunsaturated fatty acids, proteins and bioactive compounds, and immuno-stimulant effects. However, the complex polysaccharides that comprise the bulk of seaweeds' dry weight are largely indigestible by most animals. These polysaccharides may serve some function for gut health (as a probiotic, or roughage), but they may limit the utility of seaweed as an animal feed ingredient.

### SUMMARY

In an aspect, disclosed herein are methods and compositions for the microbiological pretreatment of macroalgal biomass, to create an alternative, low cost, environmentally friendly and sustainable source of key nutrients for aquaculture and live-stock industries. In an embodiment, disclosed herein is an aquatic feedstuff and ingredient quality that has no dependence on wild fishery or terrestrial resources, and in addition provides a fish-health and productivity benefit to the fish. This feed quality benefit is thought to extend to additional feed formulations, e.g. livestock (poultry, swine, etc.).

In an aspect, disclosed herein is a feed composition comprising fermented macroalgae wherein the microalgae are derived from the digestive tract of herbivorous fish. In an embodiment, the fermented macroalgae comprise up to 10 percent w/w of the feed composition.

In an embodiment, the fermented microalgae are selected from the group consisting of Rhodophyta, Phaeophyta or Chlorophyta macroalgae. In an embodiment, the fermented macroalgae is Agardhiella subulata. In an embodiment, the fermented macroalgae is Halymenia hawaiiana. In an embodiment, the fermented macroalgae is Sargassum sp. In an embodiment, the fermented macroalgae is Ulva sp. In an embodiment, the fermented macroalgae is Macrocystis sp.

In an aspect, disclosed herein is a method for improving the growth rate of fish comprising feeding fish a composition comprising fermented macroalgae wherein the microalgae are derived from the digestive tract of herbivorous fish. In an embodiment, the growth profile of the fish is improved by about 13 percent over four weeks when compared to feeding fish a composition comprising an absence of fermented macroalgae. In an embodiment, the fermented macroalgae is selected from the group consisting of any of Rhodophyta, Phaeophyta or Chlorophyta macroalgae. In an embodiment, the fermented macroalgae comprise up to 10 percent w/w of the composition. In an embodiment, the fermented macroalgae is Agardhiella subulata. In an embodiment, the fermented macroalgae is Halymenia hawaiiana. In an embodiment, the fermented macroalgae is Sargassum sp. In an embodiment, the fermented macroalgae is Ulva sp. In an embodiment, the fermented macroalgae is Macrocystis sp.

In an aspect, disclosed herein is a method for improving the growth rate of livestock comprising feeding the livestock a composition comprising fermented macroalgae wherein the microalgae are derived from the digestive tract of herbivorous fish. In an embodiment, the fermented macroalgae is selected from the group consisting of any of Rhodophyta, Phaeophyta or Chlorophyta macroalgae. In an embodiment, the fermented macroalgae comprise up to 10 percent w/w of the composition.

Other objects, advantages, and novel features of the present invention will become apparent from the following detailed description of the invention when considered in conjunction with the accompanying drawings.

### DESCRIPTION OF THE DRAWINGS

FIG. 1 depicts clockwise from top left: The 12-tank system used for the trial; tilapia being fed by hand; tilapia in tanks; juvenile tilapia; PDMA feed.

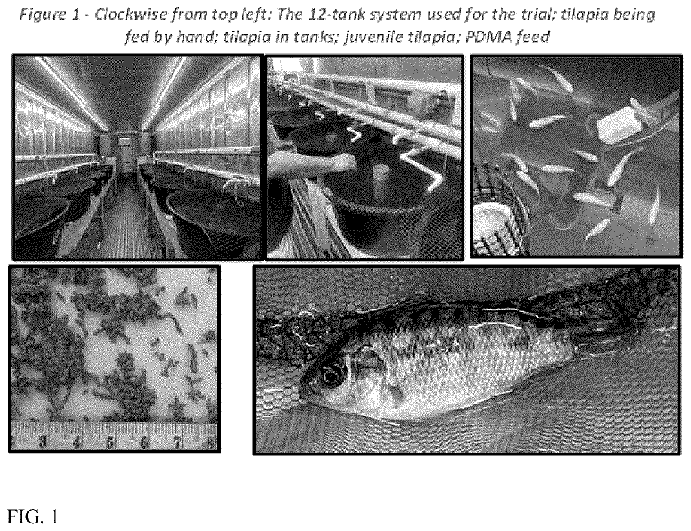

FIG. 2 depicts the mean tilapia weight (pooled for each treatment) reared on PDMA and control diets.

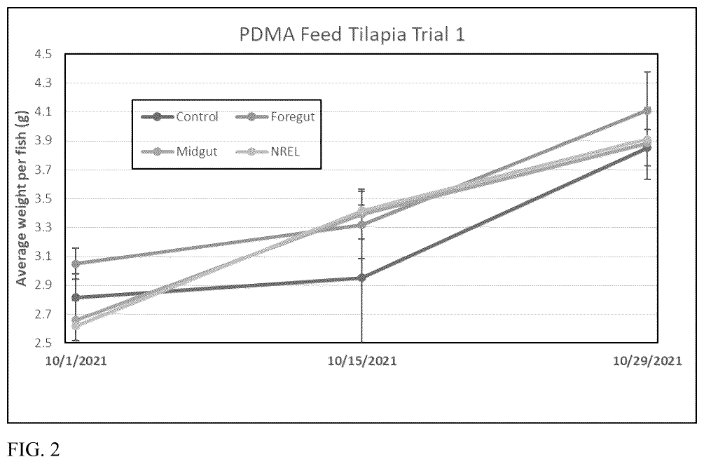

FIG. 3 depicts the percent growth increase for tilapia fed on diets formulated with PDMA inclusions over 29 days.

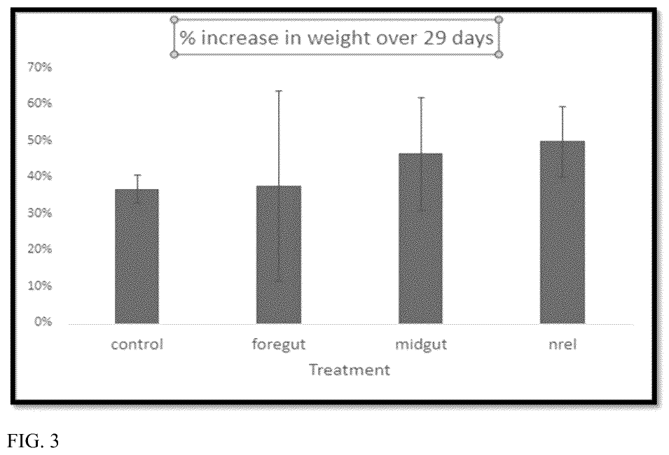

FIG. 4 depicts the composition of the solids residue after fermentation collected during the mono feed regime, showed substantial reduction in carbon content and moderate reduction in nitrogen (expressed as protein) with about 50% reduction in ash relative to the seaweed started.

FIG. 5 depicts another embodiment of the composition of the solids residue after fermentation collected during the mono feed regime, and showed substantial reduction in carbon content and moderate reduction in nitrogen (expressed as protein) with about 50% reduction in ash relative to the seaweed started.

FIG. 6 depicts a process-flow diagram of an embodiment of a PDMA feed trial and diet development.

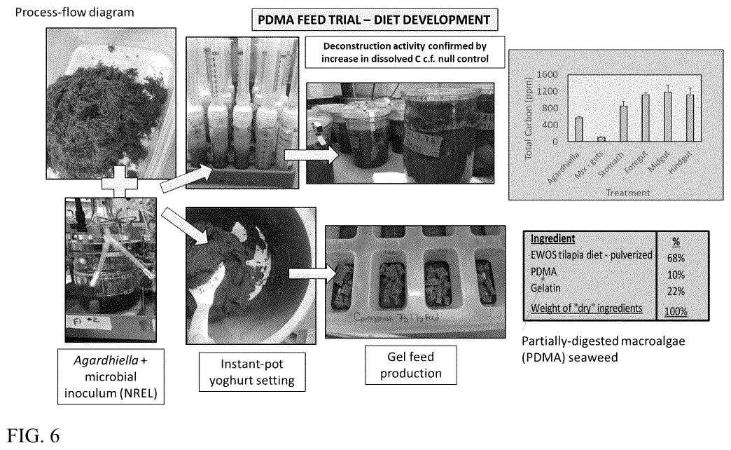

### DETAILED DESCRIPTION

Disclosed herein are methods for using and compositions of matter comprising microbially digested (with evolved microbial consortium of bacteria) seaweed or macroalgae that induce faster animal growth rates and/or may represent a more attractive or less expensive material composition which can support the decarbonization of aquaculture feed ingredients. The market size for aquaculture application that are anticipated to benefit from this novel feed ingredient is on the order of about 50 MMT global production, which at a 10% inclusion level, could open a 5 MMT market and provide associated significant carbon savings.

Disclosed herein are novel methods and compositions of matter to create a partially-digested feedstock for inclusion as a low-carbon feed ingredient alternative with a significant feed quality advantage, or a lower cost, or a lower carbon footprint.

Decarbonizing agriculture and aquaculture feed formulations requires a focus around creating ingredients that consist of lower carbon intensity compared to traditional ingredients. Macroalgae or seaweeds that are treated as described herein present an attractive, carbon-negative, feedstock which has great potential to displace some of the traditional ingredients, e.g. soybean, wheat gluten, corn, rice, and fish meal and oils. Traditional formula feeds are expensive and carbon-intensive, and represent a major operating cost in intensive fish- and livestock-rearing environments.

Aquaculture is an important sector for production of highly-nutritious food. The production of high protein food requires high-quality feed, ideally with a high (and highly digestible) protein content, which should contain not only all the necessary nutrients but also complementary additives to keep organisms healthy and stimulate growth. Improving productivity and reducing feed cost is critical for the commercial and long-term scalability of the fed-animal aquaculture industry.

Historically, macroalgae have been overlooked as a feed ingredient because of challenges associated with the high salt content and the presence of complex polysaccharides, which are often inaccessible unless the recipient species has the native microbiological activities to derive the nutritional benefits from these materials.

Disclosed herein are methods and compositions of matter to use the kyphosid (rudderfish; Kyphosidae) microbiome for biodigestion of seaweeds (macroalgae). Marine herbivores such as kyphosids appear able to extract necessary energy and protein from seaweeds. Pre-digestion of seaweeds using consortia derived from the kyphosid microbiome, or microbiome from other marine herbivorous fish, may therefore render seaweeds more digestible for other animals in aquaculture or terrestrial agriculture. This offers potential for increasing the availability of animal feedstuffs, particularly in developing countries, while reducing the economic cost and the environmental burden (in terms of CO₂ equivalents, and use of fertilizers, fresh water and arable land).

In an embodiment, disclosed herein are feed trials that evaluated growth and immune function of juvenile hybrid tilapia that were fed with diets that included 10% partially digested macroalgae (PDMA).

Four diets were tested: three experimental PDMA diets, and one control diet. The PDMAs differed by which microbial consortia was used to “pre-digest” the red algae, Agardhiella subulata. The experimental diets were based on commercially available tilapia pellets that were first pulverized, then blended with 10% PDMA and 22% gelatin. The control diet was comprised of only tilapia pellets and 22% gelatin. The feed trial was conducted in a 1000 L recirculating freshwater tank system. Fish growth, and the expression of five genes related to immune function, were evaluated.

After four weeks, the greatest increase in weight was seen in fish fed with the PDMA made with methods and compositions disclosed herein.

In an embodiment, methods for the microbiological pretreatment of macroalgal biomass are disclosed herein that create an attractive alternative, low cost, environmentally friendly and sustainable source of key nutrients for aquaculture and livestock industries. Disclosed herein are aquatic feed formulations with ingredient qualities that have a reduced fishery-derived and terrestrial feedstock resource composition and in addition may provide a fish health and productivity benefit to the fish. This feed quality benefit or lower carbon footprint or lower cost extends to additional feed formulations, e.g. livestock (poultry, swine, etc.).

The composition of the feed ingredient derived from this microbially pretreated seaweed has indicated in preliminary feed trials at Ocean Era, Kona, HI, to have a more attractive feed quality, exhibited as accelerated growth (for some PDMA forms) or comparable growth (for other PDMA forms) in Tilapia when fed on a feed formulation that includes 10% of the partially microbially-digested macroalgae feedstock.

In an embodiment, the microbial digestion may be derived from a native microbial community. In another embodiment, the microbial digestion was derived from an evolved microbial community that was tuned for more robust, and rapid, seaweed deconstruction activities over a 5-month in vivo microbial activity-based directed-evolution adaptation strategy. An aliquot of this microbial community inoculum was shipped to Ocean Era to prepare a feed ingredient, using bench-top partial pretreatment fermentation methods. This feed preparation, partially-digested seaweed material was incorporated into aquaculture feed-trials (so far only Tilapia has been tested) and demonstrated a statistically-significant increase in growth, suggesting not just a like-for-like displacement of conventional feed ingredients, but a performance boost, which was unexpected.

### Methods

### Microbes

Three different forms of PDMA were produced using three different microbial consortia: two (foregut and midgut) were from the digestive tract of wild-caught Kyphosis vaigiensis (aka Nenue or Brassy Chub), collected by spear fishing off the Kona coast. The third was from an evolved consortia comprising kyphosid gut samples and labelled as “NREL”.

### Making PDMA

Agardhiella subulata was used as the basal ingredient for all three PDMA used in this trial. Fresh A. subulata (cultured by Ocean Era) was rinsed with freshwater, and pasteurized using an Instant Pot. After pasteurization, the seaweed was added to flasks containing a sterile mixture of seawater and freshwater, adjusted to a salinity of 9 ppt. The flasks were inoculated with one of the three microbial consortia described above (foregut, midgut and NREL). These cultures were fermented for three days using Instant Pots.

### Diets

The diets were made by mixing pulverized tilapia feed pellets, gelatin and PDMA in the ratios shown in Table 1. Each experimental diet consisted 10% of PDMA, 68% pulverized commercially available tilapia diet and gelatin 22%. After combining dry ingredients and moist PDMA, boiling water was added to make a thick paste. Once the diets cooled, they were chopped into bite-sized pieces. Diets were stored in a refrigerator and were made weekly to maintain freshness.

TABLE 1
Ratios of ingredients for PDMA diet formulation:

| Ingredient | % |
|---|---|
| Pulverized tilapia pellets | 68% |
| PDMA | 10% |
| Gelatin | 22% |

### Fish Culture System

Juvenile hybrid tilapia (Oreochromis mossambicus×O. niloticus) were acquired from a hatchery in Hilo, Hawai'i. The fish were held in a freshwater recirculating system within a climate-controlled room (a converted insulated shipping container). The system consisted of 12 HDPE tanks, each 100 L volume, supplied with constant water flow and aeration. Each of the diets was fed to three replicate tanks.

The trial ran for four weeks. Fish were weighed at trial start, after two weeks, and again after four weeks, when the trial concluded. The average weight at the start of the trial was 2.8 grams.

Fish were fed twice daily to satiation. Growth performance was measured by the change in weight of each group of fish over the trial period.

In an embodiment nges to immune tion can be ascertained by using qPCR to identify possible upregulation of immune genes in the liver of individual animals.

### Results

Fish fed the diets containing the PDMA made with the NREL culture were significantly larger at trial end date (p=0.47%). The NREL-PDMA fed fish had an average weight increase of 50% over the four weeks, compared with fish fed the control diet which showed an increase in weight of 37%.

There was no significant difference in final weights between the fish fed the control diet, and those fed the diets containing the two other PDMAs (foregut and midgut). There was high variation within treatments.

TABLE 2 a
Fish weights (averaged for each tank) and percentage increase
in weight for tilapia fed the control diet and three different
PDMA diet formulations; three replicate tanks per diet:

| | | | | | Change in | |
|---|---|---|---|---|---|---|
| Diet | Replicate | Oct. 1, 2021 | Oct. 15, 2021 | Oct. 29, 2021 | wt (g) | % growth |
| Control | b | 2.7 | 2.9 | 3.7 | 1.0 | 36.9% |
| Control | c | 3.0 | 2.9 | 4.0 | 1.0 | 33.0% |
| Control | a | 2.7 | 3.1 | 3.8 | 1.1 | 40.7% |
| Foregut | a | 3.1 | 3.5 | 4.3 | 1.2 | 39.0% |
| Foregut | b | 2.5 | 3.3 | 4.1 | 1.6 | 63.2% |
| Foregut | c | 3.5 | 3.1 | 3.9 | 0.4 | 11.0% |
| Midgut | a | 2.6 | 3.7 | 4.1 | 1.5 | 56.3% |
| Midgut | b | 2.6 | 3.2 | 4.0 | 1.4 | 54.8% |
| Midgut | c | 2.8 | 3.3 | 3.6 | 0.8 | 28.6% |
| NREL | a | 2.1 | 2.8 | 3.3 | 1.2 | 56.6% |
| NREL | b | 2.8 | 3.5 | 3.9 | 1.1 | 38.9% |
| NREL | c | 2.9 | 4.0 | 4.5 | 1.6 | 54.3% |

TABLE 2 b
Fish weights (averaged for each diet treatment) and percentage
increase in weight for tilapia fed the control diet and three
different PDMA diet formulations; three replicate tanks per diet.

| | Average fish weight (grams) | | | % increase |
|---|---|---|---|---|
| Diet | Oct. 1, 2021 | Oct. 15, 2021 | Oct. 29, 2021 | in weight |
| Control | 2.82 | 2.95 | 3.85 | 36.9% |
| Foregut | 3.05 | 3.32 | 4.11 | 37.7% |
| Midgut | 2.66 | 3.39 | 3.88 | 46.6% |
| NREL | 2.62 | 3.42 | 3.91 | 50.0% |

The increased growth rate in fish fed diets containing PDMA is highly promising. The comparable growth rates of the fish fed the control diets two other PDMA are also encouraging, given the potential nutritional, economic and environmental benefits of incorporation of seaweed into animal diets.

In a prophetic embodiment, three different inclusion levels of PDMA with NREL inoculum (10%, 20%, 30%) and A. subulata, as well as a PDMA made with NREL inoculum using a different red cultivated seaweed, Halymenia hawaiiana will be tested. Additionally, chopped raw Agardhiella at a 10% inclusion rate will be used as a null control for PDMA fermentation.

Tables 3, 4, 5, 6: depict whole seaweed composition dependent upon species.

TABLE 3

| | Ash | FAME | Protein | Carbs | Uronics | Mannitol | Sum |
|---|---|---|---|---|---|---|---|
| Unknown Red | 28.4 | 0.9 | 14.9 | 33.1 | ND | ND | 77.3 |
| Sargassum Spp. | 30.9 ± 0.4 | 2.3 ± 0.4 | 12.9 ± 2.2 | 9.6 ± 0.6 | 10.8 ± 0.6 | 2.9 ± 0.8 | 69.4 ± 1.4 |
| Macrocystis pyriferia - | 38 ± 0.4 | 0.8 ± 0.3 | 3.7 ± 1.7 | 8.2 ± 0.9 | 13.5 ± 0.2 | 20 ± 4.6 | 84.2 ± 3.7 |
| Bladder | | | | | | | |
| Macrocystis pyriferia - | 31.6 ± 1.7 | 1 ± 0.3 | 4.4 ± 2.1 | 9.9 ± 2.8 | 17.2 ± 1.7 | 17.2 ± 2.8 | 81.4 ± 3.3 |
| Stipe | | | | | | | |
| Macrocystis pyriferia - | 32.1 ± 4.2 | 0.9 ± 0.1 | 5.6 ± 1.9 | 10.3 ± 0.2 | 14.2 ± 0.6 | 16.1 ± 5.9 | 79.2 ± 0.4 |
| Blade | | | | | | | |
| Sargassum and Red | 29.1 | 1.7 | 14.3 | 19.8 | 5.7 | 1.2 | 71.9 |
| Blend | | | | | | | |

TABLE 4

| | Ash | Protein | Carbohydrates | FAME |
|---|---|---|---|---|
| Sargassum | 28.8 | 6.0 | 17.5 | 1.4 |
| Agardhiella | 36.6 | 14.3 | 18.0 | 2.0 |
| Ulva | 32.2 | 6.4 | 14.4 | 1.1 |
| Macrocystis | 35.8 | 4.6 | 21.7 | 1.1 |

TABLE 5

| | Mannitol | Galactose | Glucose | Uronics |
|---|---|---|---|---|
| Sargassum | 6.6 | 1.0 | 6.4 | 10.9 |
| Agardhiella | <loq | 12.6 | 4.5 | <loq |
| Ulva | <loq | 0.2 | 5.5 | 4.0 |
| Macrocystis | 12.77 | 0.51 | 6.33 | 13.84 |

TABLE 6

| | FAME | C16:0/ | C18:1/ | C20:4/ | C20:5/ |
|---|---|---|---|---|---|
| | (% DW) | FAME | FAME | FAME | FAME |
| Sargassum | 0.1 | 36.3 | 13.1 | 11.7 | 4.5 |
| Agardhiella | 0.1 | 40.9 | 3.0 | 20.6 | 13.1 |
| Ulva | 0.2 | 52.3 | 3.3 | — | 0.5 |
| Macrocystis | 0.2 | 26.0 | 17.0 | 15.5 | 5.7 |

In an embodiment, the microbially digested seaweed residue compositional improvements include reduced ash content (˜15%), enriched lipids (3-10%)—EPA+ETA (ARA) content about 1% of the fermentation residue, and 10-18% protein content. In another embodiment, the microbially digested seaweed residue compositional improvements include reduced ash content (˜15%), enriched lipids (3-10%)—EPA+ETA (ARA) content about 1% of the fermentation residue, and up to about 40% protein content.

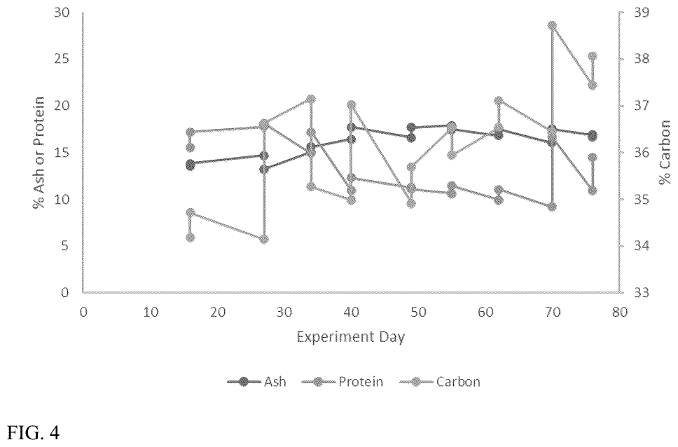

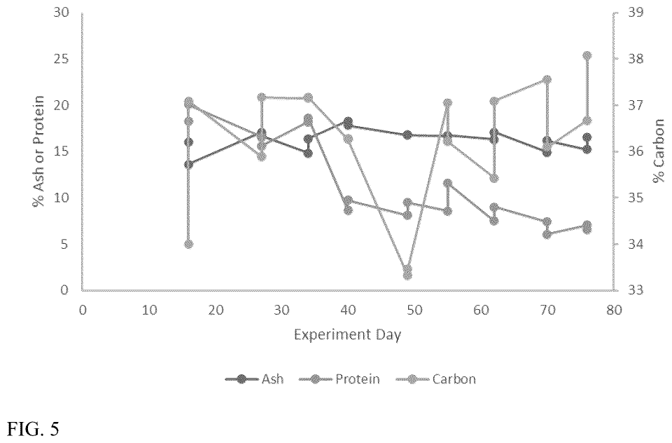

Table 7 is a depiction of the composition of the solids residue after fermentation collected during the mono feed regime and showed substantial reduction in carbon content and moderate reduction in nitrogen (expressed as protein) with about a 50% reduction in ash relative to the seaweed started (also depicted in FIGS. 4 and 5).

TABLE 7

| Sample ID | Ash | FAME | Protein* | Carbs** | Sum |
|---|---|---|---|---|---|
| GP210108-011 | 30.48 | 3.39 | 16.48 | 37.29 | 87.64 |
| Agardhiella | | | | | |
| whole biomass | | | | | |

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

What is claimed is:

1. A feed composition comprising fermented macroalgae wherein the microalgae are derived from the digestive tract of herbivorous fish.

2. The feed composition of claim 1 wherein the fermented macroalgae comprise up to 10 percent w/w of the feed composition.

3. The feed composition of claim 1 wherein the fermented microalgae are selected from the group consisting of Rhodophyta, Phaeophyta or Chlorophyta macroalgae.

4. The feed composition of claim 1 wherein the fermented macroalgae is Agardhiella subulata.

5. The feed composition of claim 1 wherein the fermented macroalgae is Halymenia hawaiiana.

6. The feed composition of claim 1 wherein the fermented macroalgae is Sargassum sp.

7. The feed composition of claim 1 wherein the fermented macroalgae is Ulva sp.

8. The feed composition of claim 1 wherein the fermented macroalgae is Macrocystis sp.

9. A method for improving the growth rate of fish comprising feeding fish a composition comprising fermented macroalgae wherein the microalgae are derived from the digestive tract of herbivorous fish.

10. The method of claim 9 wherein the growth profile of the fish is improved by about 13 percent over four weeks when compared to feeding fish a composition comprising an absence of fermented macroalgae.

11. The method of claim 9 wherein the fermented macroalgae is selected from the group consisting of any of Rhodophyta, Phaeophyta or Chlorophyta macroalgae.

12. The method of claim 9 wherein the fermented macroalgae comprise up to 10 percent w/w of the composition.

13. The method of claim 9 wherein the fermented macroalgae is Agardhiella subulata.

14. The method of claim 9 wherein the fermented macroalgae is Halymenia hawaiiana.

15. The method of claim 9 wherein the fermented macroalgae is Sargassum sp.

16. The method of claim 9 wherein the fermented macroalgae is Ulva sp.

17. The method of claim 9 wherein the fermented macroalgae is Macrocystis sp.

18. A method for improving the growth rate of livestock comprising feeding the livestock a composition comprising fermented macroalgae wherein the microalgae are derived from the digestive tract of herbivorous fish.

19. The method of claim 18 wherein the fermented macroalgae is selected from the group consisting of any of Rhodophyta, Phaeophyta or Chlorophyta macroalgae.

20. The method of claim 18 wherein the fermented macroalgae comprise up to 10 percent w/w of the composition.
